(19) United States
(12) Patent Application Publication
(10) Pub. No.: US 2017/0020366 A1
(43) Pub. Date: Jan. 26, 2017

(54) ENDOSCOPE

(71) Applicant: OLYMPUS CORPORATION, Tokyo (JP)

(72) Inventor: Takanori WATANABE, Tokyo (JP)

(73) Assignee: OLYMPUS CORPORATION, Tokyo (JP)

(21) Appl. No.: 15/288,269

(22) Filed: Oct. 7, 2016

Related U.S. Application Data
(63) Continuation of application No. PCT/JP2015/064185, filed on May 18, 2015.

(30) Foreign Application Priority Data
Aug. 6, 2014 (JP) 2014-160715

(51) Int. Cl. A61B 1/008; G02B 23/24; A61B 1/005

(52) U.S. Cl. CPC A61B 1/008; A61B 1/0055; A61B 1/0057; G02B 23/2476; G02B 23/2423

(57) ABSTRACT

An endoscope includes a bending portion configured to be bent in a plurality of directions, a plurality of joint pieces that configure the bending portion, and cylindrical-shaped wire receivers provided so as to be fixed on an inner circumferential surface or an outer circumferential surface of at least one of the plurality of joint pieces, wherein an insertion direction of a wire is tilted in a circumferential direction with respect to a center axis of the joint pieces, and a direction in which the insertion direction of the wire in one of adjacent joint pieces of the joint pieces that are bent in a same bending direction is tilted is opposite to a direction in which the insertion direction of the wire in the other of the adjacent pieces is tilted.

### CROSS REFERENCE TO RELATED APPLICATION

This application is a continuation application of PCT/JP2015/064185 filed on May 18, 2015 and claims benefit of Japanese Application No. 2014-160715 filed in Japan on Aug. 6, 2014, the entire contents of which are incorporated herein by this reference.

### BACKGROUND OF THE INVENTION

1. Field of the Invention

The present invention relates to an endoscope including, at an insertion portion to be inserted into a subject, a bending portion configured to be bent in a plurality of directions.

2. Description of the Related Art

In recent years, endoscopes have been widely used in medical fields and industrial fields. Such endoscopes are capable of performing observation, treatment and the like on a site to be examined in a subject by inserting an elongated insertion portion into the subject.

In such endoscopes, a configuration is well known in which a bending portion configured to be bendable in a plurality of directions, for example, is provided at an insertion portion of the endoscopes.

The bending portion improves the advancing property of the insertion portion at a flexing portion in a conduit, and varies an observation direction of an observation optical system provided at the distal end portion located on the front side in the longitudinal axis direction of the insertion portion (hereinafter, just referred to as “front side”) with respect to the bending portion.

Generally, the bending portion is configured to be bendable in four directions of up, down, left and right directions, for example, by coupling respective joint pieces adjacent to each other in the longitudinal axis direction of the insertion portion through rotary shafts such that the center axis of the plurality of joint pieces, each having a ring shape, is positioned in parallel with the longitudinal axis direction.

Specifically, for example, two pairs of wires, that is, four wires, which are movable forward and backward in the longitudinal axis direction, are inserted in the insertion portion so as to be opposed to each other across the center axis of the plurality of joint pieces. In addition, the distal ends in the longitudinal axis direction (hereinafter just referred to as “distal ends”) of the four wires are fixed to the joint piece located on the distal-most side in the longitudinal axis direction (hereinafter, just referred to “distal end side”) among the plurality of joint pieces. Therefore, the bending portion is bendable in any one of the up, down, left and right directions by performing pulling operation on any one of the four wires by the operation portion.

In addition, a configuration is well known, in which a wire receiver through which a wire is inserted along a longitudinal axis direction to thereby hold the wire is provided on an inner circumferential surface or an outer circumferential surface of at least one of a plurality of joint pieces.

Japanese Patent Application Laid-Open Publication No. 2009-112536 discloses a configuration in which wire receivers are provided respectively on inner circumferential surfaces of a plurality of joint pieces.

FIG. 10 is a partial cross-sectional view showing a coupling state on the design of a plurality of joint pieces at the time when the bending portion is not bent, FIG. 11 is a partial cross-sectional view showing one example of an actual coupling state of the plurality of joint pieces at the time when the bending portion is not bent, and FIG. 12 is a partial cross-sectional view showing a cross section of a wire receiver taken along XII-XII line in FIG. 11, together with a wire. Note that illustration of wires 30u, 30d is omitted in FIG. 11 in order to simplify the drawing.

As disclosed also in Japanese Patent Application Laid-Open Publication No. 2009-112536, in the state where none of the four wires is pulled by the operation portion, that is, the bending portion is not bent, when a plurality of, for example, fourteen joint pieces 20a to 20n are coupled with each other along a longitudinal axis direction N through a plurality of rotary shafts 18, and wires 30u, 30d, 30l, and 30r (wire 30r is not shown) whose distal ends are fixed to the joint piece 20a are inserted through the respective wire receivers 40ua to 40uf, 40da to 40df, 40la to 40lf, and 40ra to 40rf as shown in FIG. 10 (wire receives 40ra to 40rf are not shown), the respective joint pieces 20a to 20n do not rotate around the plurality of rotary shafts 18 on the design, and the center axis C of the respective joint pieces 20a to 20n, the penetration direction of respective wire insertion holes of the wire receivers 40ua to 40uf, 40da to 40df, 40la to 40lf, and 40ra to 40rf, and the outer circumferential surfaces and inner circumferential surfaces of the joint pieces 20a to 20n are supposed to be positioned in parallel with the longitudinal axis direction N.

That is, the respective joint pieces 20a to 20n are supposed to be positioned such that a shoulder part of the joint piece 20a and a shoulder part of the joint piece 20b, a shoulder part of the joint piece 20c and a shoulder part of the joint piece 20d, a shoulder part of the joint piece 20e and a shoulder part of the joint piece 20f, a shoulder part of the joint piece 20g and a shoulder part of the joint piece 20h, a shoulder part of the joint piece 20i and a shoulder part of the joint piece 20j, a shoulder part of the joint piece 20k and a shoulder part of the jointpiece 20l, and a shoulder part of the joint piece 20m and a shoulder part of the joint piece 20n do not abut on each other, respectively.

The wires 30u to 30r are installed in the insertion portion and the operation portion in the state where the distal ends or the respective wires 30u to 30r are fixed to the joint piece 20a and the four wires are equally pulled to the operation portion side, that is, a tensile force is equally applied to the rear side in the longitudinal axis direction (hereinafter, just referred to as rear side) of the wires.

That is, a compression force is applied to the front and back in the longitudinal axis direction (hereinafter, just referred to as front and back) of the respective joint pieces 20a to 20n by the wires 30u to 30r.

According to the compression force, in the state where the bending portion is not bent, the respective joint pieces 20a to 20n are not actually in the state as shown in FIG. 10, and as shown in FIG. 11, the center axis C of the respective joint pieces 20b to 20m, and the penetration direction of the respective wire insertion holes 40uai to 40ufi, 40dai to 40dfi, 40lai to 40lfi, and 40rai to 40rfi (wire insertion holes 40rai to 40th are not shown) of the respective wire receivers 40ua to 40uf, 40da to 40df, 40la to 40lf, and 40ra to 40rf, which is coincident with the center axis C, is tilted with respect to the longitudinal axis direction N.

That is, the joint pieces 20a to 20n are positioned such that the shoulder part of the joint piece 20a and the shoulder part of the joint piece 20b, the shoulder part of the joint piece 20c and the shoulder part of the joint piece 20d, the shoulder part of the joint piece 20e and the shoulder part of the joint piece 20f, the shoulder part of the joint piece 20g and the shoulder part of the joint piece 20h, the shoulder part of the joint piece 20i and the shoulder part of the joint piece 20j, the shoulder part of the joint piece 20k and the shoulder part of the joint piece 20l, and the shoulder part of the joint piece 20m and the shoulder part of the joint piece 20n abut on each other, respectively, which causes a backlash to occur in the joint pieces.

Note that the backlash state of the joint pieces 20a to 20f shown in FIG. 11 is just one example, and the form of backlash is not limited to the example. In addition, the form of backlash in the joint pieces 20a to 20f is also related to the balance of rigidities of the various internal components provided in the bending portion. Furthermore, it is known that the backlash state in the joint pieces 20a to 20f is produced regardless of the kind and alignment of the joint pieces, the method of fixing the wire receivers and rivets, and the like.

### SUMMARY OF THE INVENTION

An endoscope according to one aspect of the present invention includes: a bending portion provided at an insertion portion to be inserted into a subject, the bending portion being configured to be bent in a plurality of directions; a plurality of joint pieces that configure the bending portion, each of which is formed in a ring shape, the plurality of joint pieces having a center axis positioned along a longitudinal axis direction of the insertion portion, wherein pieces adjacent to each other along the longitudinal axis direction are coupled with each other respectively through rotary shafts; and cylindrical-shaped wire receivers through which wires for bending the bending portion are respectively inserted, the wire receivers being provided so as to be fixed on an inner circumferential surface or an outer circumferential surface of at least one of the plurality of joint pieces, wherein an insertion direction of each of the wires into each of the wire receivers provided respectively on adjacent joint pieces is tilted in a circumferential direction of each of the wire receivers with respect to the center axis of the plurality of joint pieces, the adjacent joint pieces being among the plurality of joint pieces and bent in a same bending direction, and a direction in which the insertion direction of each of the wires into the wire receiver provided on one of the adjacent joint pieces is tilted is set to be opposite to a direction in which the insertion direction of each of the wires into the wire receiver provided on the other of the adjacent joint pieces is tilted.

### BRIEF DESCRIPTION OF THE DRAWINGS

FIG. 1 illustrates an appearance of an endoscope according to a first embodiment.

FIG. 2 is a partial cross-sectional view showing a coupling state of joint pieces provided in a bending portion in FIG. 1 and a state where an extending direction of a wire is restricted by wire receivers at the time when the bending portion is not bent.

FIG. 3 is a cross-sectional view showing a modified example in which a wire insertion hole is formed in a wire receiver such that only the wire insertion hole is tilted with respect to a center axis in a state where the bending portion shown in FIG. 1 is not bent and a backlash occurs in a plurality of joint pieces.

FIG. 4 is a partial cross-sectional view showing a state where the wire is inserted through the wire insertion hole shown in FIG. 3.

FIG. 5 is a partial cross-sectional view showing a coupling state of joint pieces provided in a bending portion according to a second embodiment and a state where an extending direction of a wire is restricted by wire receivers at the time when the bending portion is not bent.

FIG. 6 is a partial cross-sectional view showing a coupling state of the joint pieces provided in the bending portion and a state where the extending direction of the wire is restricted by the wire receivers at the time when the bending portion is not bent in a state where a position of each of the wire receivers is shifted in a radial direction from the position shown in FIG. 11.

FIG. 7 is a partial cross-sectional view showing a state where four-direction pieces and two-direction pieces are alternately coupled with each other in the plurality of joint pieces.

FIG. 8 is a partial cross-sectional view showing a state where a plurality of joint pieces configured by two-direction pieces are coupled with each other.

FIG. 9 illustrates a cross section of the bending portion.

FIG. 10 is a partial cross-sectional view showing a coupling state on the design of a plurality of joint pieces at the time when the bending portion is not bent.

FIG. 11 is a partial cross-sectional view showing one example of an actual coupling state of the plurality of joint pieces at the time when the bending portion is not bent.

FIG. 12 is a partial cross-sectional view showing a cross section of the wire receiver taken along XII-XII line in FIG. 11, together with the wire.

### DETAILED DESCRIPTION OF THE PREFERRED EMBODIMENT(S)

Hereinafter, embodiments of the present invention will be described.

### FIRST EMBODIMENT

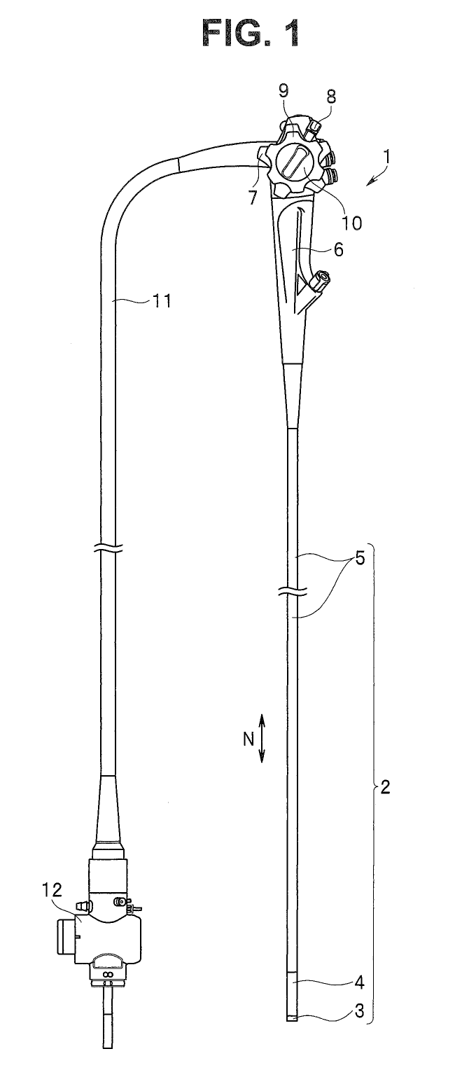

FIG. 1 illustrates an appearance of an endoscope according to the present embodiment.

As shown in FIG. 1, an endoscope 1 includes a main part configured by including an insertion portion 2 configured to be inserted in a subject, an operation portion 6 provided continuously with the proximal end side in a longitudinal axis direction N (hereinafter, just referred to as proximal end side) of the insertion portion 2, a universal cord 11 extended from the operation portion 6, and a connector 12 provided at an extension end of the universal cord 11. Note that the endoscope 1 is electrically connected to external apparatuses such as a control apparatus and an illumination apparatus, through a connector 12.

The operation portion 6 is provided with an up/down bending operation knob 7 for bending a bending portion 4, to be described later, of the insertion portion 2 in up and down directions, and a left/right bending operation knob 9 for bending the bending portion 4 in left and right directions.

Furthermore, the operation portion 6 is provided with a fixing lever 8 that fixes the rotation position of the up/down bending operation knob 7 and a fixing knob 10 that fixes the rotation position of the left/right bending operation knob 9.

The insertion portion 2 is configured by including a distal end portion 3, the bending portion 4, and a flexible tube portion 5 in this order from the distal end side, and formed in an elongated shape.

The bending portion 4 is bent in a plurality directions, for example, four directions of up, down, left and right directions, by rotation operation of the up/down bending operation knob 7 and the left/right operation knob 9, to thereby change an observation direction of an observation optical system, not shown, provided in the distal end portion 3, and improve the insertion performance of the distal end portion 3 in a subject. Furthermore, the flexible tube portion 5 is provided continuously with the proximal end side of the bending portion 4.

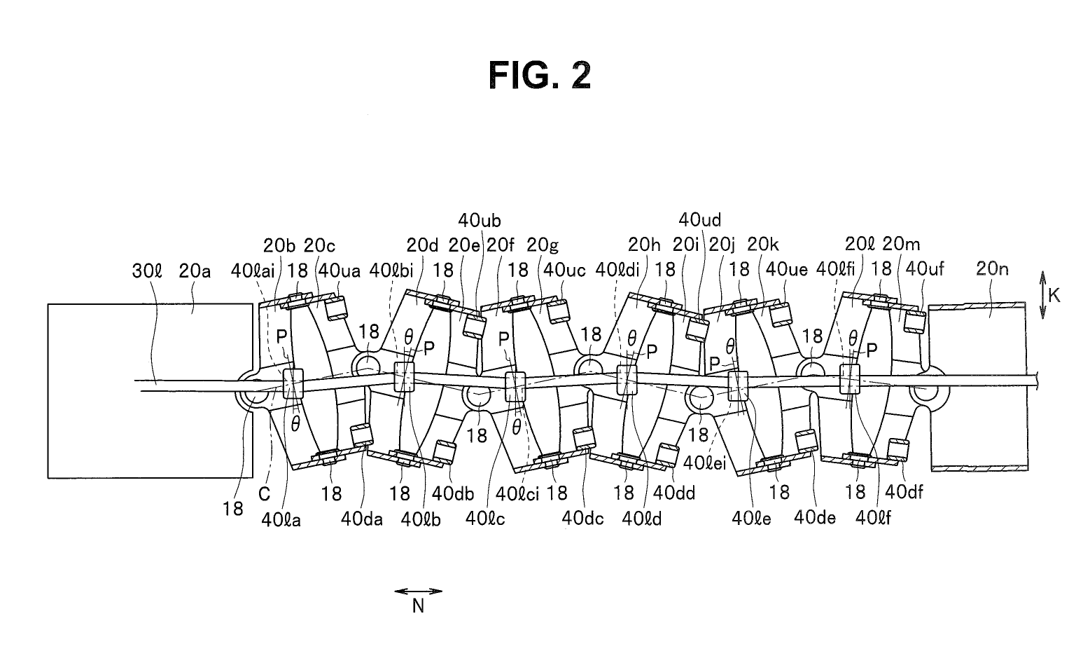

Next, the configuration in the bending portion 4 will be described with reference to FIG. 2. FIG. 2 is a partial cross-sectional view showing a coupling state of joint pieces provided in the bending portion in FIG. 1 and a state where an extending direction of a wire is restricted by wire receivers at the time when the bending portion is not bent.

As shown in FIG. 2, the bending portion 4 includes, inside thereof, a plurality of joint pieces 20a, 20b, 20c, 20d, 20e, 20f, 20g, 20h, 20i, 20j, 20k, 20l, 20m and 20n, which configure the bending portion 4 and each of which is formed in a ring shape.

The plurality of joint pieces 20a to 20n have a center axis C positioned along the longitudinal axis direction N of the insertion portion, and are configured by four-direction pieces bendable in the four directions by the respective pieces adjacent to each other being coupled with each other through rotary shafts 18.

In addition, inside the insertion portion 2 and the operation portion 6, two pairs of wires which are configured to bend the bending portion 4 and which are opposed to each other across the center axis C, that is, four wires 30u, 30d, 30l, and 30r (the wire 30r is not shown, and the wires 30u, 30d are omitted in FIG. 2 for simplifying the drawing) are inserted, and the distal ends of the respective wires 30u to 30r are fixed to the joint piece 20a.

Note that, though not shown, the proximal end of the wire 30u and the proximal end of the wire 30d are coupled with one end and the other end of an up/down chain wound around the sprocket of the up/down bending operation knob 7, respectively, in the operation portion 6, and the proximal end of the wire 30l and the proximal end of the wire 30r are coupled with one end and the other end of a left/right chain wound around the sprocket of the left/right bending operation knob 9, respectively, in the operation portion 6.

According to such a configuration, when the up/down bending operation knob 7 is rotated in one direction, the wire 30u is pulled, to thereby cause the bending portion 4 to be bent in the up direction, and when the up/down bending operation knob 7 is rotated in the other direction, the wire 30d is pulled, to thereby cause the bending portion 4 to be bent in the down direction.

When the left/right bending operation knob 9 is rotated in one direction, the wire 30l is pulled, to thereby cause the bending portion 4 to be bent in the left direction, and when the left/right bending operation knob 9 is rotated in the other direction, the wire 30r is pulled, to thereby cause the bending portion 4 to be bent in the right direction.

In addition, as shown in FIG. 2, wire receivers 40ua, 40ub, 40uc, 40ud, 40ue, and 40uf, wire receivers 40da, 40db, 40dc, 40dd, 40de, and 40df, wire receivers 40la, 40lb, 40lc, 40ld, 40le, and 40lf, and wire receivers 40ra, 40rb, 40rc, 40rd, 40re, and 40rf (the wire receivers 40ra to 40rf are not shown) through which the wires 30u to 30r are inserted respectively, are provided on the inner circumferential surfaces of the joint pieces 20b to 20m.

Specifically, the wire receivers 40la and 40ra are provided on the inner circumferential surface of the joint piece 20b so as to be opposed to each other across the center axis C, the wire receivers 40ua and 40da are provided on the inner circumferential surface of the joint piece 20c so as to be opposed to each other across the center axis C, the wire receivers 40lb and 40rb are provided on the inner circumferential surface of the joint piece 20d so as to be opposed to each other across the center axis C, the wire receivers 40ub and 40db are provided on the inner circumferential surface of the joint piece 20e so as to be opposed to each other across the center axis C, the wire receivers 40lc and 40rc are provided on the inner circumferential surface of the joint piece 20f so as to be opposed to each other across the center axis C, and the wire receivers 40uc and 40dc are provided on the inner circumferential surface of the joint piece 20g so as to be opposed to each other across the center axis C. Furthermore, the wire receivers 40ld and 40rd are provided on the inner circumferential surface of the joint piece 20h so as to be opposed to each other across the center axis C, the wire receivers 40ud and 40dd are provided on the inner circumferential surface of the joint piece 20i so as to be opposed to each other across the center axis C, the wire receivers 40le and 40re are provided on the inner circumferential surface of the joint piece 20j so as to be opposed to each other across the center axis C, the wire receivers 40ue and 40de are provided on the inner circumferential surface of the joint piece 20k so as to be opposed to each other across the center axis C, the wire receivers 40lf and 40rf are provided on the inner circumferential surface of the joint piece 20l so as to be opposed to each other across the center axis C, and the wire receivers 40uf and 40df are provided on the inner circumferential surface of the joint piece 20m so as to be opposed to each other across the center axis C.

Note that the respective wire receivers 40ua to 40uf, 40da to 40df, 40la to 40lf, and 40ra to 40rf may be respectively provided on the outer circumferential surfaces of the joint pieces 20b to 20m.

In addition, the respective wire receivers 40ua to 40uf include wire insertion holes 40uai to 40ufi for the wire 30u, not shown, which are formed in parallel with the center axis C, the respective wire receivers 40da to 40df include wire insertion holes 40dai to 40dfi for the wire 30d, not shown, which are formed in parallel with the center axis C, the respective wire receivers 40la to 40lf include wire insertion holes 40lai, 40lbi, 40lci, 40ldi, 40lei, and 40lfi for the wire 30l which are formed in parallel with the center axis C, and the respective wire receivers 40ra to 40rf include wire insertion holes 40rai to 40rfi for the wire 30r, not shown, which are formed in parallel with the center axis C.

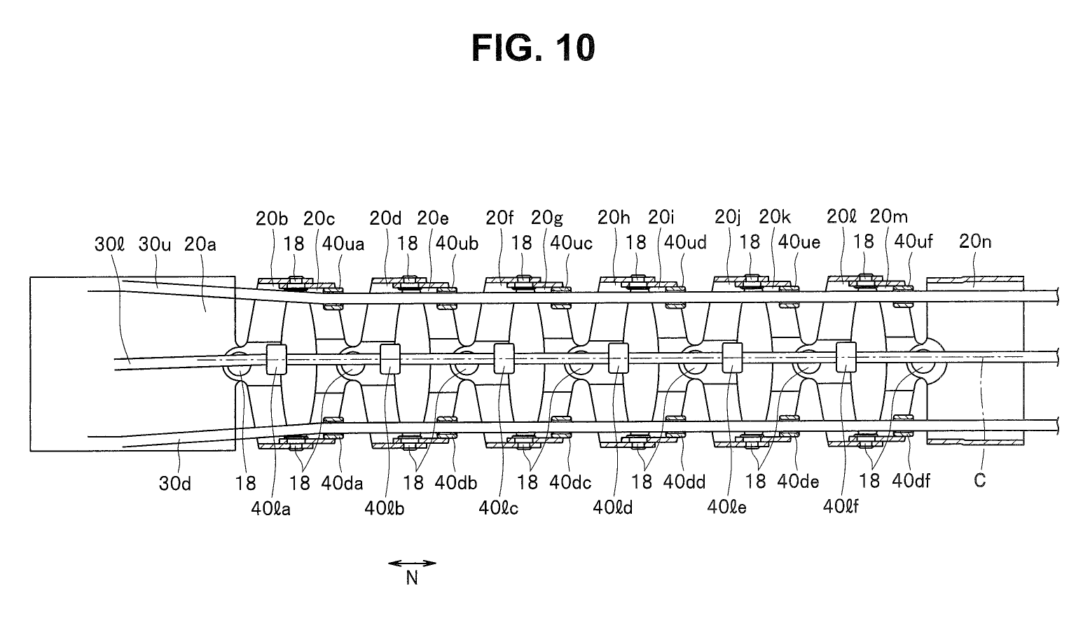

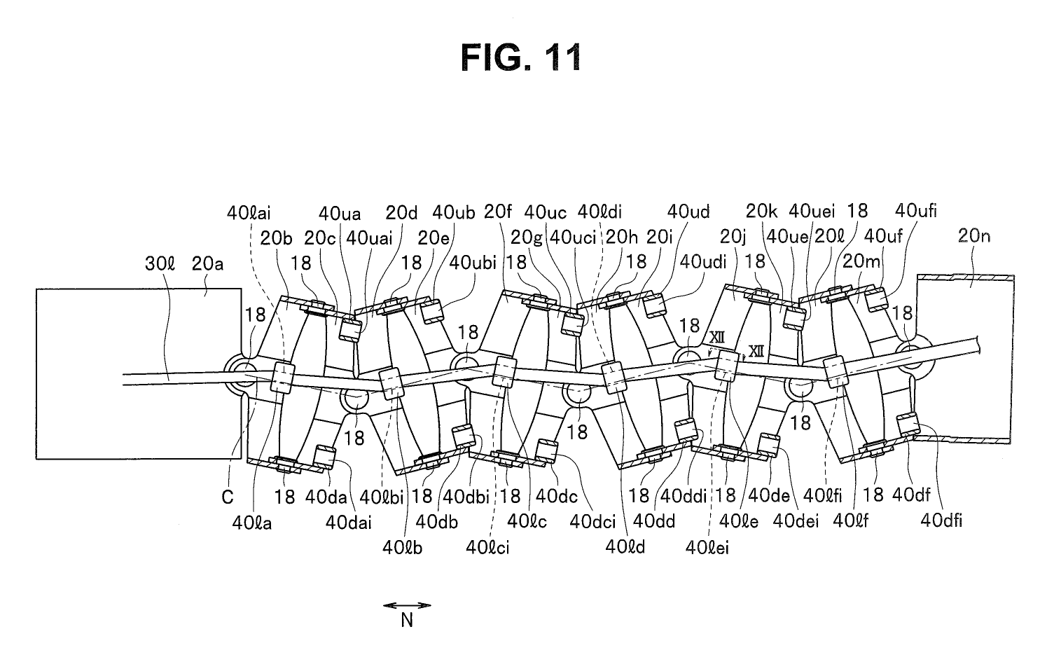

As described above, when the bending portion 4 is not bent as shown in FIG. 1, the respective joint pieces 20a to 20n are not in the state as shown in FIG. 10, and the wires 30u to 30r are equally pulled rearward, to thereby cause a compression force along the longitudinal axis direction N to be generated on the joint pieces 20a to 20n as described above. As a result, the center axis C of the joint pieces 20a to 20m and the penetration direction of the wire insertion holes 40uai to 40ufi, 40dai to 40dfi, 40lai to 40lfi, and 40rai to 40rfi are located so as to be tilted with respect to the longitudinal axis direction N, as shown in FIG. 11.

That is, a backlash occurs by the joint pieces being arranged such that the shoulder part of the joint piece 20a and the shoulder part of the joint piece 20b, the shoulder part of the joint piece 20c and the shoulder part of the joint piece 20d, the shoulder part of the joint piece 20e and the shoulder part of the joint piece 20f, the shoulder part of the joint piece 20g and the shoulder part of the joint piece 20h, the shoulder part of the joint piece 20i and the shoulder part of the joint piece 20j, the shoulder part of the joint piece 20k and the shoulder part of the jointpiece 20l, and the shoulder part of the joint piece 20m and the shoulder part of the joint piece 20n abut on each other, respectively, which causes meandering of the wires 30u to 30r, as shown in FIG. 11.

Hereinafter, with reference to FIG. 2, description will be made on the configuration of the present embodiment for suppressing the meandering of the wires 30u to 30r even in the case where the backlash occurs in the joint pieces 20a to 20n in the state where the bending portion 4 is not bent as shown in FIG. 1, by taking the wire 30l and the wire receivers 40la to 40lf as an example.

As shown in the above-described FIG. 11, when the wire receivers 40la to 40lf are provided on the joint pieces 20b, 20d, 20f, 20h, 20j and 20l such that the wire insertion holes 40lai to 40lfi are positioned in parallel with the center axis C and the inner circumferential surfaces and the outer circumferential surfaces of the joint pieces 20b to 20m, if the backlash occurs in the joint pieces 20a to 20f, the wire insertion holes 40lai to 40lfi are tilted with respect to the longitudinal axis direction N. As a result, the wire 30l meanders.

In order to address such a circumstance, in the present embodiment, when the backlash occurs in the joint pieces 20a to 20n in the state where the bending portion 4 is not bent, the wire receivers 40la to 40lf are provided respectively on the joint pieces 20b, 20d, 20f, 20h, 20j, and 20l such that the insertion direction of the wire 30l into each of the wire receivers 40la to 40lf is tilted with respect to the center axis C, as shown in FIG. 2.

Specifically, the wire receivers 40la to 40lf are provided respectively on the joint pieces 20b, 20d, 20f, 20h, 20j, and 20l so as to be tilted together with the wire insertion holes 40lai to 40lfi by θ with respect to the center axis C such that the wire insertion holes 40lai to 40lfi of the wire receivers 40la to 40lf are in parallel with the longitudinal axis direction N. In other words, the wire receivers 40la to 40lf are provided so as to be tilted together with the wire insertion holes 40lai to 40lfi by θ with respect to the axis P which is orthogonal to the longitudinal axis direction N. That is, θ is an angle at which the wire receivers 40la to 40lf are tilted such that the wire insertion holes 40lai to 40lfi are brought in parallel with the longitudinal axis direction N. Therefore, the angle θ varies depending on the tilt angle of the joint pieces 20a to 20n when the backlash occurs in each of the joint pieces 20a to 20n.

As a result, when the backlash occurs in the joint pieces 20a to 20n in the state where the bending portion 4 is not bent, the wire 30l meanders more or less, but the meandering angle is small, as shown in FIG. 2. As a result, the wire 30l is inserted through each of the wire insertion holes 40lai to 40lfi so as to be in parallel with the longitudinal axis direction N. The meandering of the wire 30l is thus suppressed in the present embodiment.

The above-described configuration is similarly applied to the wire receivers 40ua to 40uf, 40da to 40df, and 40ra to 40rf provided on the joint pieces 20b to 20m, though not shown. That is, the wire receivers 40ua to 40uf, 40da to 40df, and 40ra to 40rf are provided so as to be tilted by the angle θ with respect to the joint pieces 20b to 20m such that the wire insertion holes 40uai to 40ufi, 40dai to 40dfi, 40rai to 40rfi are tilted with respect to the center axis C, to be brought in parallel with the longitudinal axis direction N.

Thus, in the present embodiment, when the backlash occurs in the joint pieces 20a to 20n in the state where the bending portion 4 is not bent, each of the wire receivers 40ua to 40uf, 40da to 40df, 40la to 40lf, and 40ra to 40rf is provided so as to be tilted by the angle θ with respect to each of the joint pieces 20b to 20m such that the insertion direction of each of the wires 30u to 30r into each of the wire receivers 40ua to 40uf, 40da to 40df, 40la to 40lf, and 40ra to 40rf is tilted with respect to the center axis C, specifically, each of the wire insertion holes 40uai to 40ufi, 40dai to 40dfi, 40lai to 40lfi, and 40rai to 40rfi is also tilted with respect to the center axis C, to be brought in parallel with the longitudinal axis direction N.

According to such a configuration, even if the backlash occurs in the joint pieces 20a to 20n, the wires 30u to 30r are inserted respectively through the wire insertion holes 40uai to 40ufi, 40dai to 40dfi, 40lai to 40lfi, and 40rai to 40rfi so as to be in parallel with the longitudinal axis direction N, which enables the meandering of the wires 30u to 30r to be suppressed, and the wires 30u to 30r are inserted through the wire insertion holes 40uai to 40ufi, 40dai to 40dfi, 40lai to 40lfi, and 40rai to 40rfi in a substantially non-contact state with respect to the wire receivers 40ua to 40uf, 40da to 40df, 40la to 40lf, and 40ra to 40rf, which reduces the friction to be applied to the wires 30u to 30r, compared with the state shown in FIG. 11. As a result, the durability of the wires 30u to 30r can be improved.

Thus, the present embodiment can provide the endoscope including the wire receivers configured to be capable of improving the durability of the wires for bending the bending portion.

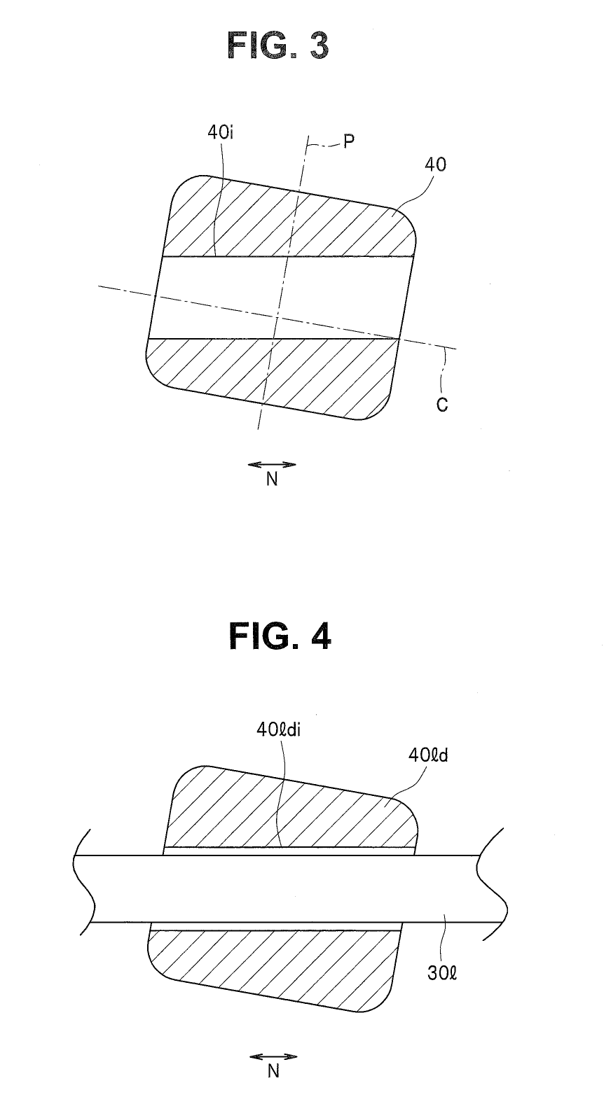

Note that a modified example will be described with reference to FIGS. 3 and 4. FIG. 3 is a cross-sectional view showing a modified example in which a wire insertion hole is formed in a wire receiver such that only the insertion hole is tilted with respect to the center axis in the state where the bending portion as shown in FIG. 1 is not bent and a backlash occurs in a plurality of joint pieces. FIG. 4 is a partial cross-sectional view showing the state where the wire is inserted in the wire insertion hole shown in FIG. 3.

In the above-described present embodiment, when the backlash occurs in the joint pieces 20a to 20n in the state where the bending portion 4 is not bent, the wire receivers 40ua to 40uf, 40da to 40df, 40la to 40lf, and 40ra to 40rf are provided so as to be tilted together with the wire insertion holes 40uai to 40ufi, 40dai to 40dfi, 40lai to 40lfi, and 40rai to 40rfi by the angle θ with respect to the joint pieces 20b to 20m, in order to cause the insertion direction of each of the wires 30u to 30r into each of the wire receivers 40ua to 40uf, 40da to 40df, 40la to 40lf, and 40ra to 40rf to be tilted with respect to the center axis C.

The present invention is not limited to such a configuration, and only the wire insertion hole 40i formed in the wire receiver 40 may be tilted with respect to the center axis C, as shown in FIG. 3, in order to cause the insertion direction of each of the wires 30u to 30r into each of the wire receivers 40ua to 40uf, 40da to 40df, 40la to 40lf, and 40ra to 40rf to be tilted with respect to the center axis C.

Note that the configuration of the wire receiver 40 is applied to the wire receivers 40ua to 40uf, 40da to 40df, 40la to 40lf, and 40ra to 40rf, and the configuration of the wire insertion hole 40i is applied to the wire insertion holes 40uai to 40ufi, 40dai to 40dfi, 40lai to 40lfi, and 40rai to 40rfi.

Specifically, even if the wire receiver 40 is provided on the joint pieces 20b to 20m as shown in the above-described FIG. 11, only the wire insertion hole 40i may be formed tilted with respect to the center axis C so as to be in parallel with the longitudinal axis direction N as shown in FIG. 3.

According to such a configuration, as shown in FIG. 4, when the backlash occurs in the joint pieces 20a to 20n in the state where the bending portion 4 is not bent, the wires 30u to 30r are inserted through the wire insertion hole 40i so as to be in parallel with the longitudinal axis direction N, similarly as shown in FIG. 2. As a result, the present modified example can provide the same effects as those in the present embodiment.

### SECOND EMBODIMENT

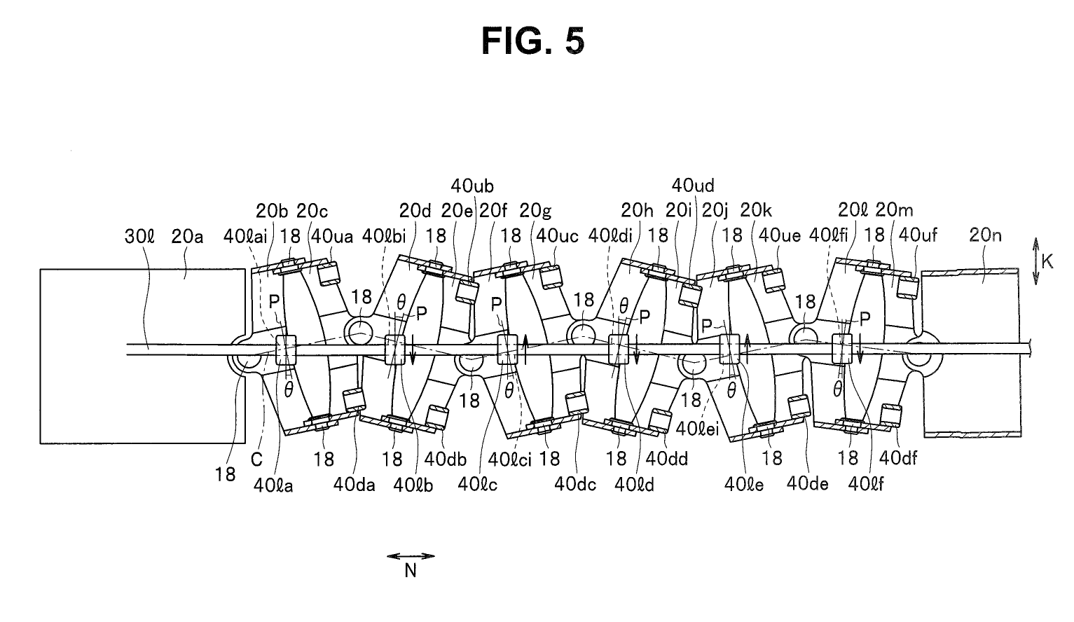

FIG. 5 is a partial cross-sectional view showing a coupling state of the joint pieces provided in the bending portion according to the present embodiment and the state where the extending direction of the wire is restricted by the wire receivers at the time when the bending portion is not bent.

The configuration of the endoscope according to the second embodiment is different from the endoscope according to the first embodiment shown in the above-described FIG. 1 to FIG. 4 in that the wire receivers are positioned not only tilted with respect to the center axis but also shifted along the radial direction of the plurality of joint pieces. Therefore, only the different point will be described and the same constituent elements as those in the first embodiment are attached with the same reference numerals and descriptions thereof will be omitted.

Note that, in FIG. 5, illustration of the wires 30u and 30d is omitted for simplifying the drawing also in the present embodiment, and description is made on the configuration according to the present embodiment in which the meandering to be generated on the wires 30u to 30r is suppressed even when the backlash occurs in the joint pieces 20a to 20n in the state where the bending portion 4 is not bent as shown in FIG. 1, by taking the wire 30l and the wire receivers 40la to 40lf as an example.

In the above-described first embodiment, when the backlash occurs in the joint pieces 20a to 20n in the state where the bending portion 4 is not bent, in order to cause the insertion direction of each of the wires 30u to 30m into each of the wire receivers 40ua to 40uf, 40da to 40df, 40la to 40lf, and 40ra to 40rf to be tilted with respect to the center axis C, as shown in FIG. 2, the wire receivers 40ua to 40uf, 40da to 40df, 40la to 40lf, and 40ra to 40rf are provided so as to be tilted together with the wire insertion holes 40uai to 40ufi, 40dai to 40dfi, 40lai to 40lfi, and 40rai to 40rfi by the angle θ with respect to the joint pieces 20b to 20m, or as shown in FIG. 3 and FIG. 4, only the wire insertion hole 40i is formed tilted with respect to the center axis C so as to be in parallel with the longitudinal axis direction N.

In addition, in the present embodiment, as shown in FIG. 5, the wire receivers 40lb to 40lf are positioned on the joint pieces 20d, 20f, 20h, 20j, and 20l so as to be shifted in the radial direction K of the joint pieces 20a to 20n from the state where the outer circumferential surfaces and the inner circumferential surfaces of the joint pieces 20a to 20n are in parallel with the longitudinal axis direction N as shown in FIG. 10.

Specifically, the wire receiver 40lb is positioned on the joint piece 20d so as to be shifted in the lower direction in FIG. 5 from the position in the radial direction K as shown in FIG. 11 and FIG. 2, the wire receiver 40lc is positioned on the joint piece 20f so as to be shifted in the upper direction in FIG. 5 from the position in the radial direction K as shown in FIG. 11 and FIG. 2, the wire receiver 40ld is positioned on the joint piece 20h so as to be shifted in the lower direction in FIG. 5 from the position in the radial direction K as shown in FIG. 11 and FIG. 2, the wire receiver 40le is positioned on the joint piece 20j so as to be shifted in the upper direction in FIG. 5 from the position in the radial direction K as shown in FIG. 11 and FIG. 2, and the wire receiver 40lf is positioned on the jointpiece 20l so as to be shifted in the lower direction in FIG. 5 from the position in the radial direction K as shown in FIG. 11 and FIG. 2.

The above-described configuration is similarly applied to the wire receivers 40ua to 40uf, 40da to 40df, and 40ra to 40rf provided on the joint pieces 20b to 20m, though not shown.

Other configurations are the same as those in the above-described first embodiment.

In the configuration according to the first embodiment, when the backlash occurs in the joint pieces 20a to 20n in the state where the bending portion 4 is not bent, the wires 30u to 30r are inserted through the wire insertion holes 40uai to 40ufi, 40dai to 40dfi, 40lai to 40lfi, and 40rai to 40rfi, respectively, so as to be in parallel with the longitudinal axis direction N. However, the wires 30u to 30r meander more or less, as shown in FIG. 2.

However, the configuration of the present embodiment is capable of surely preventing the wires 30u to 30r from meandering due to the arrangement in which the positions of the wire receivers 40ua to 40uf, 40da to 40df, 40ra to 40rf are shifted in the radial direction K from the positions shown in FIG. 2 until the wires 30u to 30r becomes parallel with the longitudinal axis direction N.

Therefore, the wires 30u to 30r are inserted through the wire insertion holes 40uai to 40ufi, 40dai to 40dfi, 40lai to 40lfi, and 40rai to 40th, respectively, so as to have less contact with the wire receivers than in the first embodiment. That is, the present embodiment is capable of more surely preventing the wires 30u to 30r from being abraded due to the contact with the wire receivers 40ua to 40uf, 40da to 40df, and 40ra to 40rf, compared with the above-described first embodiment. Note that other effects are the same as those in the above-described first embodiment.

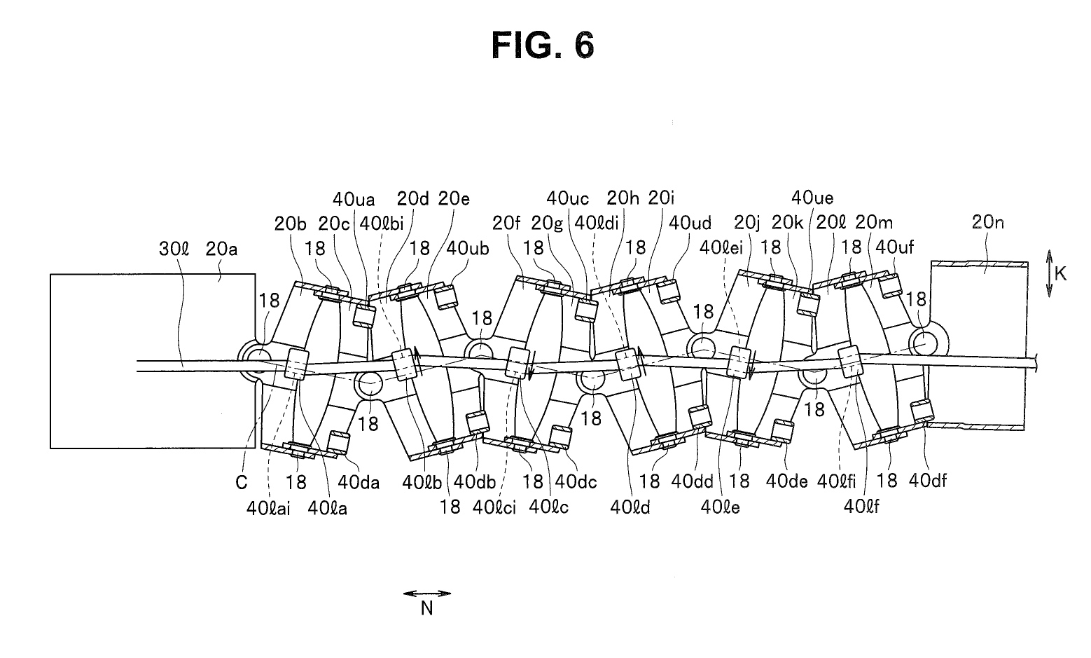

Note that a modified example is described with reference to FIG. 6. FIG. 6 is a partial cross-sectional view showing a coupling state of the joint pieces provided in the bending portion and the state where the extending direction of the wire is restricted by the wire receivers at the time when the bending portion is not bent in the state where the position of each of the wire receivers is shifted in the radial direction from the position shown in FIG. 11.

Note that illustration of the wires 30u and 30d is omitted also in FIG. 6 for simplifying the drawing also in the present modified example, and description is made on the configuration in which the meandering to be generated in the wires 30u to 30r is suppressed when the backlash occurs in the joint pieces 20a to 20n in the state where the bending portion 4 is not bent as shown in FIG. 1, by taking the wire 30l and the wire receivers 40la to 40lf as an example.

In the above-described present embodiment, when the backlash occurs in the joint pieces 20a to 20n in the state where the bending portion 4 is not bent, in order to cause the insertion direction of each of the wires 30u to 30r into each of the wire receivers 40ua to 40uf, 40da to 40df, 40la to 40lf, and 40ra to 40rf to be tilted with respect to the center axis C, the wire receivers 40ua to 40uf, 40da to 40df, 40la to 40lf, and 40ra to 40rf are provided so as to be tilted together with the wire insertion holes 40uai to 40ufi, 40dai to 40dfi, 40lai to 40lfi, and 40rai to 40rfi by the angle θ with respect to the joint pieces 20b to 20m, as shown in FIG. 5, and the wire receivers 40ua to 40uf, 40da to 40df, 40la to 40lf, and 40ra to 40rf are positioned on the joint pieces 20b to 20m so as to be shifted in the radial direction K of the joint pieces 20a to 20n from the positions shown in FIG. 5.

However, the present embodiment is not limited to the configuration, and as shown in FIG. 6, the wire receivers 40ua to 40uf, 40da to 40df, 40la to 40lf, and 40ra to 40rf may be positioned on the joint pieces 20d, 20f, 20h, 20j, and 20l so as to be shifted from the positions shown in FIG. 11 and FIG. 2 in the radial direction K of the joint pieces 20a to 20n to the positions shown in FIG. 5 and FIG. 6, without tilting the insertion direction of each of the wires 30u to 30r into each of the wire receivers 40ua to 40uf, 40da to 40df, 40la to 40lf, and 40ra to 40rf with respect to the center axis C, that is, in the state where the insertion direction is coincident with the center axis C.

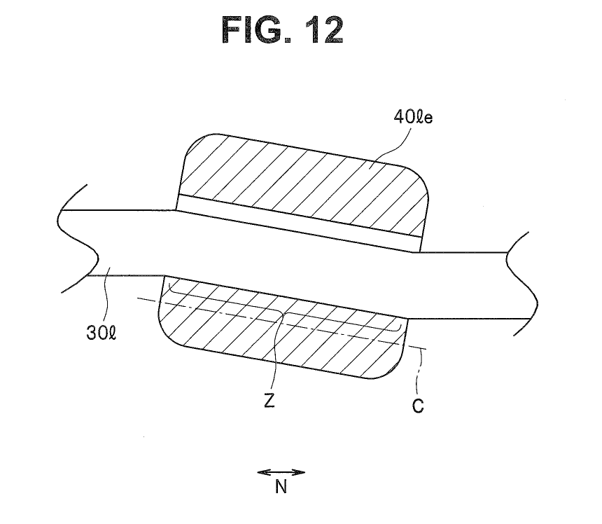

In this case, as shown in FIG. 6, the wire insertion holes 40uai to 40ufi, 40dai to 40dfi, 40lai to 40lfi, and 40rai to 40rfi are tilted together with the center axis C with respect to the longitudinal axis direction N, and the wires 30u to 30r passing respectively through the wire insertion holes 40uai to 40ufi, 40dai to 40dfi, 40lai to 40lfi, and 40rai to 40rfi are likely to be brought into contact with the wire receivers 40ua to 40uf, 40da to 40df, 40la to 40lf, and 40ra to 40rf, similarly as in the configurations shown in FIG. 11 and FIG. 12. However, since the wire receivers 40lb to 40lf are positioned shifted in the radial direction K, the meandering of the wires 30u to 30r is more suppressed than in the configuration shown in FIG. 11. Therefore, the present modified example can prevent the abrasion of the wires 30u to 30r and improve the durability of the wires more effectively than in the configuration shown in FIG. 11, though the present modified example is slightly less excellent in the effectiveness than the present embodiment and the first embodiment.

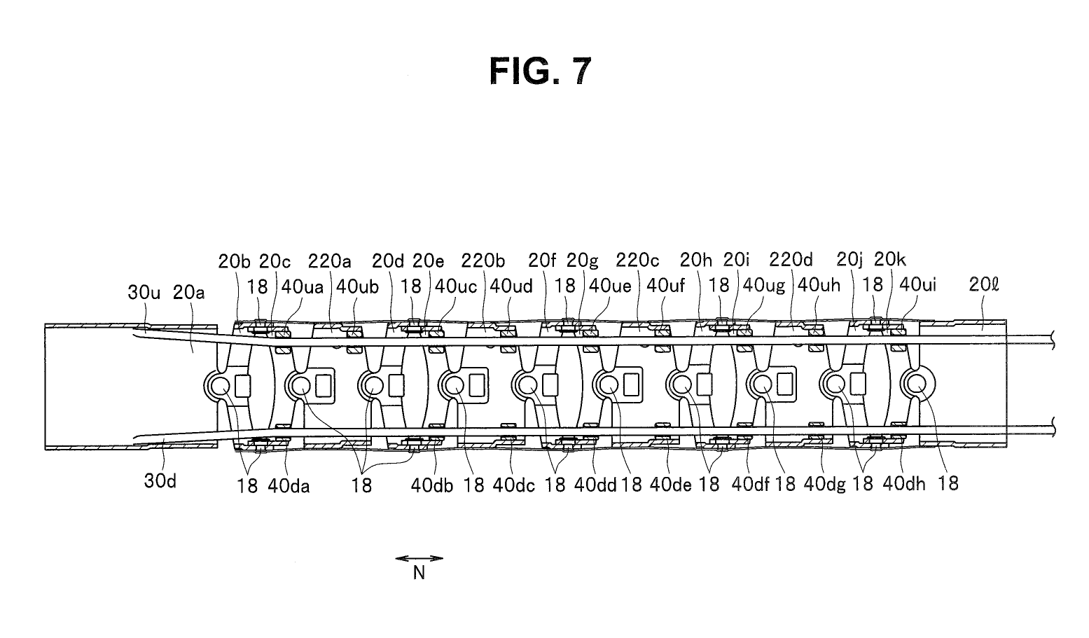

Note that, hereinafter, another modified example is described with reference to FIG. 7. FIG. 7 is a partial cross-sectional view showing the state where four-direction pieces and two-direction pieces are alternately coupled with each other in the plurality of joint pieces.

In the first and second embodiments, a plurality of joint pieces are configured by the four-direction pieces 20a to 20n that are bendable in four directions being coupled with each other along the longitudinal axis direction N through the rotary shafts 18.

However, the present invention is not limited to the configuration, and the four-direction pieces and the two-direction pieces that are bendable in two directions may be coupled with each other alternately.

Specifically, as shown in FIG. 7, the joint piece 20a as the four-direction piece is coupled with the joint piece 20b as the four-direction piece through the rotary shaft 18, the joint piece 20b is coupled with the joint piece 20c as the four-direction piece through the rotary shaft 18, the joint piece 20c is coupled with a joint piece 220a as the two-direction piece through the rotary shaft 18, and the joint piece 220a is coupled with the joint piece 20d as the four-direction piece through the rotary shaft 18.

In addition, the joint piece 20d is coupled with the joint piece 20e as the four-direction piece through the rotary shaft 18, the joint piece 20e is coupled with a joint piece 220b as the two-direction piece through the rotary shaft 18, and the joint piece 220b is coupled with the joint piece 20f as the four-direction piece through the rotary shaft 18.

Furthermore, the joint piece 20f is coupled with the joint piece 20g as the four-direction piece through the rotary shaft 18, the joint piece 20g is coupled with a joint piece 220c as the two-direction piece through the rotary shaft 18, and the joint piece 220c is coupled with the joint piece 20h as the four-direction piece through the rotary shaft 18.

In addition, the joint piece 20h is coupled with the joint piece 20i as the four-direction piece through the rotary shaft 18, the joint piece 20i is coupled with the joint piece 220d as the two-direction piece through the rotary shaft 18, the joint piece 220d is coupled with the joint piece 20j as the four-direction piece through the rotary shaft 18, the joint piece 20j is coupled with the joint piece 20k as the four-direction piece through the rotary shaft 18, and the joint piece 20k is coupled with the joint piece 20l as the four-direction piece through the rotary shaft 18.

Also in such a configuration, the above-described first and second embodiments are also applicable.

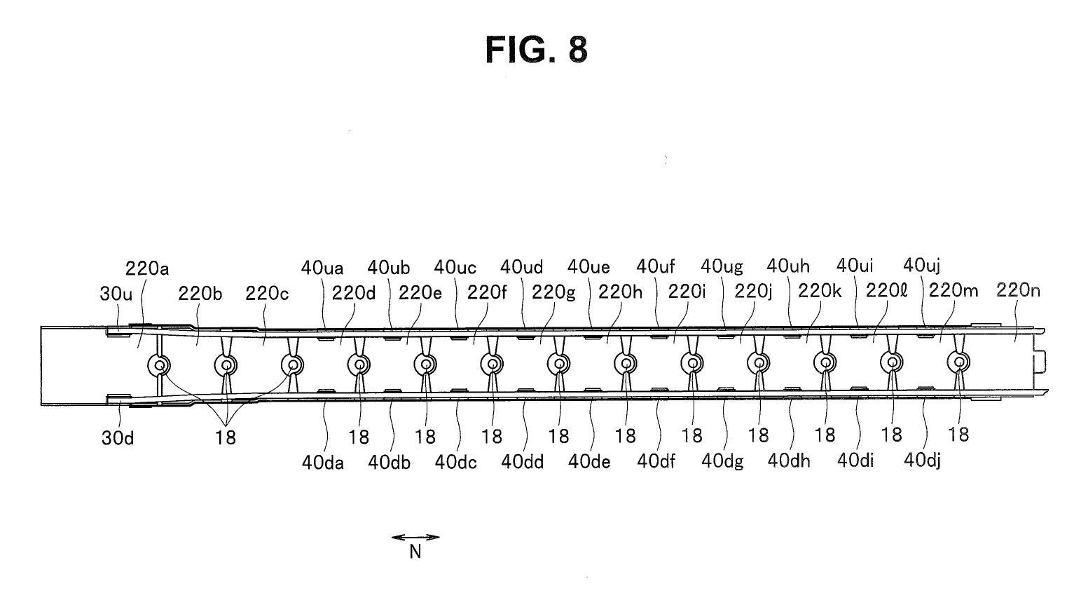

Hereinafter, another modified example is described with reference to FIG. 8. FIG. 8 is a partial cross-sectional view showing the state where a plurality of joint pieces, which are configured by two-direction pieces, are coupled with each other.

In the above-described first and second embodiments, the plurality of joint pieces that configure the bending portion 4 are constituted of the joint pieces 20a to 20n that are four-direction pieces bendable in four directions. That is, the bending portion 4 is bendable in four directions.

The present invention is not limited to the configuration, and the plurality of joint pieces that configure the bending portion 4 may be constituted of joint pieces 220a to 220n that are two-direction pieces bendable in two directions and the pieces adjacent to each other may be coupled with each other along the longitudinal axis direction N through the respective rotary shafts 18. That is, the bending portion 4 may be configured to be bendable in two directions. Note that FIG. 8 illustrates the bending portion 4 by taking the configuration in which the bending portion 4 is bendable only in up and down directions as an example. However, it is needless to say that the bending portion 4 may be configured to be bendable only in left and right directions.

Also in such a configuration, the above-described first and second embodiments are also applicable.

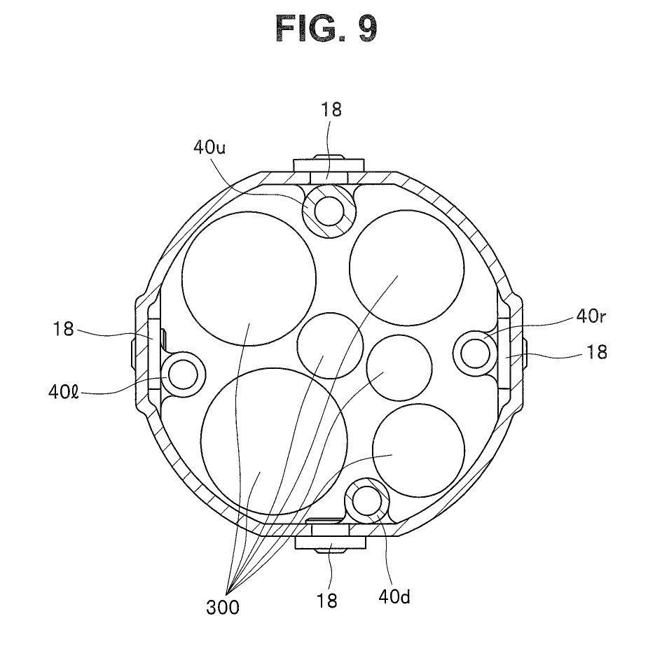

Furthermore, another modified example is described with reference to FIG. 9. FIG. 9 illustrates a cross section of the bending portion.

As shown in FIG. 9, the wire receiver 40u (corresponding to the wire receivers 40ua to 40uf), the wire receiver 40d (corresponding to the wire receivers 40da to 40df), the wire receiver 40l (corresponding to the wire receivers 40la to 40lf), and the wire receiver 40r (corresponding to the wire receivers 40ra to 40rf) may have different sizes.

In addition, the wire receiver 40r may be formed integrally with the rotary shaft 18, for example, as shown in FIG. 9. It is needless to say that also the wire receivers 40u, 40d, and 40l may be formed integrally with the rotary shafts 18.

Furthermore, as shown in FIG. 9, the wire receivers 40d, 40l may be configured not to be opposed to the wire receivers 40u, 40r, and may be positioned shifted from the rotary shafts 18, for example.

Also in such a configuration, the above-described first and second embodiments are also applicable.

In addition, the lengths in the longitudinal axis direction N of the wire receivers 40ua to 40uf, 40da to 40d, 40la to 40lf and 40ra to 40rf may be set to be greater in the above-described first and second embodiments.

Such a configuration allows the interference lengths in the longitudinal axis direction N of the wire receivers 40ua to 40uf, 40da to 40d, 40la to 40lf, and 40ra to 40rf with respect to the wires 30u to 30r to be greater, which allows a restraining force applied from the wire receivers 40ua to 40uf, 40da to 40d, 40la to 40lf, and 40ra to 40rf for causing the wires 30u to 30r to be extended in parallel with the longitudinal axis direction N to be stronger.

Furthermore, in the first and second embodiments, description has been made by taking the case where the wire receivers through which the wires 30u to 30r are inserted are provided and the six wire receivers are provided for each of the wires 30u to 30r as an example. The number of the wire receivers provided for each of the wires, however, is not limited to six, and it is needless to say that one or any number of wire receivers may be provided for each of the wires.

The present invention is thus capable of providing the endoscope having the wire receivers configured to be capable of improving the durability of the wires for bending the bending portion

The present invention is not limited to the above-described embodiments, and various changes, modifications, and the like are possible without changing the gist of the present invention.

What is claimed is:

1. An endoscope comprising:
a bending portion provided at an insertion portion to be inserted into a subject, the bending portion being configured to be bent in a plurality of directions;
a plurality of joint pieces that configure the bending portion, each of which is formed in a ring shape, the plurality of joint pieces having a center axis positioned along a longitudinal axis direction of the insertion portion, wherein pieces adjacent to each other along the longitudinal axis direction are coupled with each other respectively through rotary shafts; and
cylindrical-shaped wire receivers through which wires for bending the bending portion are respectively inserted, the wire receivers being provided so as to be fixed on an inner circumferential surface or an outer circumferential surface of at least one of the plurality of joint pieces,
wherein an insertion direction of each of the wires into each of the wire receivers provided respectively on adjacent joint pieces is tilted in a circumferential direction of each of the wire receivers with respect to the center axis of the plurality of joint pieces, the adjacent joint pieces being among the plurality of joint pieces and bent in a same bending direction, and a direction in which the insertion direction of each of the wires into the wire receiver provided on one of the adjacent joint pieces is tilted is set to be opposite to a direction in which the insertion direction of each of the wires into the wire receiver provided on the other of the adjacent joint pieces is tilted.

2. The endoscope according to claim 1, wherein in at least one of the plurality of joint pieces, a center axis of each of the wire receivers is tilted together with an insertion hole for each of the wires in the circumferential direction with respect to the center axis of the plurality of joint pieces, the insertion hole being formed in each of the wire receivers.

3. The endoscope according to claim 1, wherein only the insertion hole for each of the wires, which is formed in each of the wire receivers, is tilted with respect to the center axis of the plurality of joint pieces.

4. The endoscope according to claim 1, wherein each of the wire receivers provided respectively on the adjacent joint pieces is arranged so as to be shifted in the circumferential direction with respect to each of the rotary shafts of the plurality of joint pieces, and a direction in which an arranging position of the wire receiver provided on one of the adjacent pieces is shifted is opposite to a direction in which an arranging position of the wire receiver provided on the other of the adjacent pieces is shifted.

5. The endoscope according to claim 1, wherein when the bending portion is in a non-bent state, the wires are extended in such a manner that one of at least one pair of the wires is opposed to the other across the center axis, with the at least one pair of the wires being equally pulled to a rear side in the longitudinal axis direction.
